(19) United States
(12) Patent Application Publication
(10) Pub. No.: US 2004/0111135 A1
(43) Pub. Date: Jun. 10, 2004

(54) Photodynamic therapy apparatus with solid-state laser and therapy method using the same

(76) Inventor: Jongwon Kim, Daejeon City (KR)

Correspondence Address: JENKENS & GILCHRIST, PC, 1445 ROSS AVENUE, SUITE 3200, DALLAS, TX, 75202, US

(21) Appl. No.: 10/315,482

(22) Filed: Dec. 10, 2002

(51) Int. Cl. A61N 5/067

(52) U.S. Cl. 607/89

(57) ABSTRACT

The present invention relates to a photodynamic therapy (PDT) apparatus and a therapy method using the same, and the PDT apparatus comprises a laser beam generator for generating laser beams of a predetermined wavelength necessary for curing a tumor, which includes a pulse pumping lamp for generating the light of a predetermined vibration energy and a laser stick for generating laser beams of a predetermined wavelength by using the light from the pulse pumping lamp; a power supply for providing the laser beam generator with power; a first optical resonance reflecting mirror of convex shape for reflecting and amplifying the laser beams of the predetermined wavelength from the laser beam generator; a first rotation reflecting mirror for receiving and reflecting again the laser beams reflected from the first optical resonance; a barium nitrate crystal (Ba(NO3)2) for receiving the laser beams reflected from the first rotation reflecting mirror and converting the wavelength of the received laser beams; a lithium fluoride (Lif) crystal for converting again the wavelength of the converted laser beams from the barium nitrate crystal; a KTP (Potassium Tytanil Phosphate; KTiOPO4) crystal for outputting the laser beams of the converted wavelength passed from the lithium fluoride crystal at a predetermined wavelength necessary for curing a tumor: a second and a third rotation reflecting mirror for reflecting only the laser beams of a predetermined wavelength in the laser beams from the KTP crystal; and a focusing lens for condensing the light reflected from the third rotation reflecting mirror and irradiating the light to tumor cells through an optical fiber cable directly or with an endoscope, thereby curing only a tumor without necrotizing normal texture by injecting HematoPorfilin Derivative (HPD) to a patient and applying laser beams to the tumor.

### BACKGROUND OF THE INVENTION

(1) Field of the Invention

The present invention relates to a photodynamic therapy (PDT) apparatus and a therapy method using the same, more particularly to cure only a tumor (cancer) without necrotizing normal texture by injecting HematoPorfilin Derivative (HPD), a kind of blood components, to a patient and applying laser beams to the tumor.

(2) Description of the Related Arts

In the conventional optical therapy method for curing tumor, the HPD more accumulated in tumor texture than normal texture is injected into a patient through his vein and gold vapor laser beams of wavelength 628 nm is applied to the tumor part (the diseased part) in which the HPD is accumulated, so that the tumor part is activated by color laser beams and singlet oxygen, which is a kind of cytotoxin, is produced by a photodynamic reaction of the HPD. This singlet oxygen destroys the tumor texture so that the tumor is cured.

Generally, the blood component HPD has the utility bandwidth of wavelength 440˜450 nm. Within the blood of a human body, the HPD operates in wavelength 605˜630 nm, where the first absorption bandwidth is 630 nm and lasers has been developed to suit the bandwidth. The metal vapor laser of the lasers is used because of generating automatically the bandwidth. On the other hand, a solid-state crystal makes a bandwidth of wavelength 608 nm that is the second absorption bandwidth.

In case of using the metal vapor laser, however, after reacting on metal vapor completely, the tube of the metal vapor laser should be replaced consecutively with a new. In case of using crystal as the solid-state laser, a semi-permanent apparatus free from a crack is provided. Further, in the case of a vapor or liquid laser, it takes one or two hours to warm up, which is the time for preheating the tube. The solid-state laser, however, generates pulses simultaneous with applying power.

As an optical therapy method by making use of above described principle, there is a perfect reaction for completely necrotizing the tumor, which is started within 24 hours after a beam treatment and confirmed with a clot capable of promoting in a beam field or by a biopsy. Generally, it means that the tumor is cured with developing into the necrosis within 24 hours after the beam treatment.

Further, typical medicinal substances for the conventional optical therapy are photofrin, phtogam and the like. As an apparatus for generating a red laser beam, a helium/neon laser for generating beams of wavelength 632.8 nm or an adjustable dye laser for diverging between wavelength 631˜635 nm may be used as substitute for an arc lamp.

On the other hand, an injection syringe and optical fibers are used as a tumor therapy apparatus with a laser. In this case, after local anesthesia, a tumor part is ascertained by supersonic waves. Next, the injection syringe is injected and the optical fibers connected with a dividing apparatus are inserted into the injection syringe so that the ends of the optical fibers protrude at about 2 mm from the injection syringe to the outside. Under above condition, a laser generating apparatus generates laser beams. The generated laser beams are divided into several places(usually, four places) by the dividing apparatus and irradiated into the tumor part at a predetermined period through each optical fiber, thereby necrotizing the tumor in a predetermined range (whose diameter is about 5 cm).

In case of curing the tumor with above therapy apparatus, however, it is not completely recovered from metastatic cancers because the cancers are necrotized locally. Further, since a nidus is not precisely ascertained by supersonic waves, above therapy apparatus is not applied to focal cancers. Moreover, if it is bad at curing, then normal texture neighboring to the tumor may be necrotized too.

### SUMMARY OF THE INVENTION

It is therefore an object of the present invention to provide a photodynamic therapy apparatus and a therapy method using the same capable of solving the problems with the prior art.

To achieve the above-mentioned objectives, according to the present invention, there is provided a photodynamic therapy apparatus with a solid-state laser and a therapy method using the same, which is able to cure even focal cancers by injecting HematoPorfilin Derivative (HPD) to a patient and applying laser beams to the cancers.

The photodynamic therapy apparatus using a solid-state laser comprising: a laser beam generator for generating laser beams of a predetermined wavelength necessary for curing a tumor, which includes a pulse pumping lamp for generating the light of a predetermined vibration energy and a laser stick for generating laser beams of a predetermined wavelength by using the light from the pulse pumping lamp; a power supply for providing the laser beam generator with power; a first optical resonance reflecting mirror of convex shape for reflecting and amplifying the laser beams of the predetermined wavelength from the laser beam generator; a first rotation reflecting mirror for receiving and reflecting again the laser beams reflected from the first optical resonance; a barium nitrate crystal (Ba(NO3)2) for receiving the laser beams reflected from the first rotation reflecting mirror and converting the wavelength of the received laser beams; a lithium fluoride (Lif) crystal for converting again the wavelength of the converted laser beams from the barium nitrate crystal; a KTP (Potassium Tytanil Phosphate; KTiOPO4) crystal for outputting the laser beams of the converted wavelength passed from the lithium fluoride crystal at a predetermined wavelength necessary for curing a tumor: a second and a third rotation reflecting mirror for reflecting only the laser beams of a predetermined wavelength in the laser beams from the KTP crystal; and a focusing lens for condensing the light reflected from the third rotation reflecting mirror and irradiating the light to tumor cells through an optical fiber cable directly or with an endoscope.

The photo-dynamic therapy method using a solid state laser comprising the steps of: injecting HPD (HematoProfilin Derivative) which is a kind of blood component into the tumor texture of a patient; removing the HPD of the normal texture, while accumulating the HPD of the tumor texture; applying laser beams of wavelength 608 nm to the tumor texture by using the photo-dynamic therapy apparatus; generating active oxygen by reacting the HPD with the laser beams chemically; and necrotizing the tumor texture by destroying the lipid of the tumor texture with the active oxygen.

Therefore, according to the photodynamic therapy apparatus and the therapy method related to the present invention, after injecting the HPD through the vein of a patient and accumulating the HPD in the tumor part, the tube of a standard endoscope is inserted into the part to be cured and the laser beam generator, which is a kind of pump, is operated. The laser beams generated by the laser beam generator are irradiated to the tumor part through the optical fiber so that the HPD is activated. By the activating of the HPD, oxygen within the tumor is activated and comes out to the outside, so that the tumor is necrotized. It follows that the tumor is detected accurately and cured easily.

### BRIEF DESCRIPTION OF THE DRAWINGS

FIG. 1 is a schematic illustration of the photodynamic therapy apparatus according to the present invention.

### DETAILED DESCRIPTION OF THE EMBODIMENTS

In the following, embodiments of the present invention will be described with reference the attached drawings.

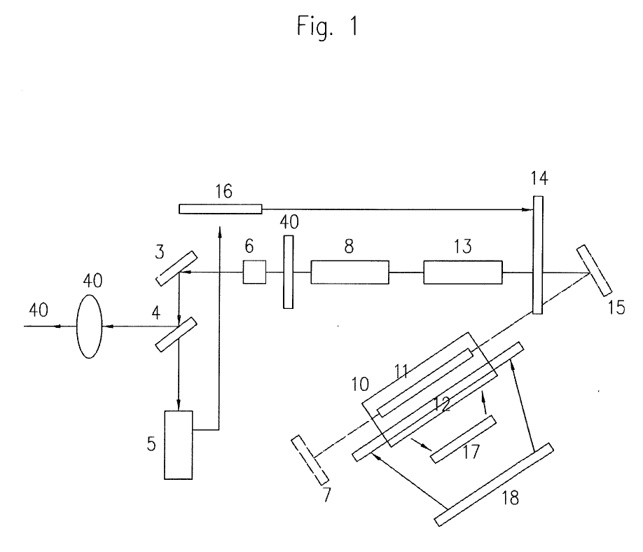

As shown in FIG. 1, the photodynamic therapy (PDT) apparatus using a solid-state laser, according to the present invention, comprises a laser beam generator 10, which is a kind of pump, for generating laser beams of a predetermined wavelength necessary for curing a tumor, which includes a pulse pumping lamp 12 such as a pulse flash lamp for generating the light of a predetermined vibration energy and an a laser stick 11, a kind of active device, for generating laser beams of a predetermined wavelength by using the light from the pulse pumping lamp; a power supply 18 for providing the laser beam generator with power; a cooler 17 for preventing the pulse pumping lamp from overheating; a first optical resonance reflecting mirror 7 of convex shape for reflecting and amplifying the laser beams of the predetermined wavelength from the laser beam generator; a first rotation reflecting mirror 15 for receiving and reflecting again the laser beams reflected from the first optical resonance; a barium nitrate crystal (Ba(NO3)2) 13 for receiving the laser beams reflected from the first rotation reflecting mirror and converting the wavelength of the received laser beams by using Ramman Scattering; a Q-switching lithium fluoride (Lif) crystal 9 for converting again the wavelength of the converted laser beams from the barium nitrate crystal through Q-switching; a second optical resonance reflecting mirror 8 for receiving the laser beams from the lithium fluoride crystal and reflecting the laser beams of the non-converted wavelength while outputting the laser beams of the converted wavelength; a non-linear KTP (Potassium Tytanil Phosphate; KTiOPO4) crystal 6 for outputting the laser beams of the converted wavelength passed from the second optical resonance reflecting mirror at a predetermined wavelength: a second and a third rotation reflecting mirror 3, 4 for reflecting only the laser beams of a predetermined wavelength in the laser beams from the non-linear KTP crystal; an absorption energy meter 5 for absorbing a portion of the laser beams passed through the third rotation reflecting mirror 3 and measuring the energy of the absorbed laser beams; a central processor 16 for receiving the output from the absorption energy meter and controlling the whole operation of the photo-dynamic therapy apparatus; a safety shutter 14 for excluding the laser beams reflected from the fifth rotation reflecting mirror under the controlling of the central processor in case that the laser beams measured by the absorption energy meter deviates from a predetermined limit; and a focusing lens 2 for condensing the light reflected from the third rotation reflecting mirror and irradiating the light to tumor cells through an optical fiber cable directly or with an endoscope.

Hereinafter, the operation of the PDT therapy apparatus for curing a tumor according to the present invention will be described.

First, the power supply 18 provides the pulse-pumping lamp 12 such as a pulse flash lamp with power, so that the pulse-pumping lamp generates the light of a pulse type. The laser stick 11, a kind of active device, receives the light and generates laser beams. As for the laser stick 11, YAG:Nd⁺³ or YAP:Nd⁺³ is used, where the YAG:Nd⁺³ generates laser beams whose wavelength are 1064 nm and the YAP:Nd⁺³ generates laser beams whose wavelength are 1079 nm. Although the YAP:Nd⁺³ will be described as an example, the present invention is not limited to the case.

The first optical resonance reflecting mirror 7 of a convex shape is arranged in one end of the laser stick, which reflects, amplifies and excites the light of semi-laser state generated in the laser stick. The laser beams from the laser stick travel to the first rotation reflecting mirror 15 whose reflection coefficient R is 99.9% at the wavelength 1060˜1220 nm. The laser beams, which are reflected and amplified by the first optical resonance reflecting mirror 7 and reflected again from the laser stick to the first rotation reflecting mirror 15, pass through the barium nitrate crystal 13. The laser beams of wavelength 1079 nm passed through the barium nitrate crystal are converted into the wavelength 1079 nm by Ramman Scattering, and pass through the Q-switching lithium fluoride crystal 9. The laser beams of wavelength 1079 nm passed through the Q-switching lithium fluoride crystal are converted pulse waves by the Q-switching of wavelength 1216 nm. The converted laser beams travel to the second optical resonance-reflecting mirror 8. Only the converted laser beams of wavelength 1216 nm are passed through the second optical resonance-reflecting mirror 8, while the unconverted laser beams of wavelength 1079 nm are excluded. The laser beams passed through the second optical resonance reflecting mirror 8 are converted into the wavelength 608 nm by passing through the non-linear KTP crystal 6. With passing through the second and third rotation reflecting mirror 3, 4, the unconverted laser beams of wavelength 1216 nm are excluded from the converted laser beams, so that only the laser beams of wavelength 608 nm are output. The output laser beams is condensed through the focusing lens 2, and irradiated into tumor cells of a patient through an optical fiber cable directly or with an endoscope.

In the PDT apparatus using a solid-state laser according to the present invention, further, the third rotation-reflecting mirror passes the laser beams of about 1%. The energy meter 5 receives the passed laser beams of 1%, measures the intensity of the laser beams, and outputs the measured intensity to the central processor 16. Therefore, if the laser beams output through the focusing lens 2 are deviates from a predetermined safety range so large as to affect not only the tumor part but also the normal part, the energy meter 5 measures the intensity and outputs the measured intensity to the central processor 6. Then, the central processor 6 starts the safety shutter 14 according to the program stored in advance, so that the laser beams are excluded. As described above, since the energy meter 5 always measures the intensity of the laser beams, though outputting excessively, the safety shutter 14 excludes the laser beams. Therefore, it prevents the normal part of a patient from damaging due to the excessive irradiating of the laser beams.

Probably, the range of the laser output capable of curing is 200 mW˜1W, and the average of the output is 500 mW.

The central processor controls the safety shutter and connects with the optical parts and the cooler of the PDT apparatus. The central processor detects all errors occurred in the power supply, the optical parts and the cooler, and starts the safety shutter excluding the laser beams in case of detecting an error. That is to say, when the shutter is started when the laser beams are generated, and the excluding threshold is about 1W.

Hereinafter, the PDT method for using the PDT apparatus according to the present invention is described.

First, in case of curing the tumor (cancer) of a patient with the PDT apparatus; HPD (HematoProfilin Derivative), which is a kind of blood component and more accumulated in tumor texture than normal texture, is injected into the patient through his vein. After a predetermined time, the injected HPD has lost its efficacy or is discharged from the patient's body, but remains in only the part of the tumor because of more accumulating in that.

Next, the PDT apparatus using the solid-state laser of YAP:Nd⁺³ generates the laser beams of wavelength 608 nm and irradiates the beams into the tumor part in which the HPD is accumulated. Then, the HPD reacts on chemically by the laser beams of the wavelength 608 nm so that active oxygen is generated. The active oxygen destroys the lipid of the tumor texture, thereby necrotizing the tumor.

Hereinafter, the producing and reaction of the active oxygen is described briefly. Being provided with the sufficient energy (ATP) to live, a human being is chiefly dependent on the metabolism of carbohydrate and fat. The carbohydrate generates more energy in case that oxygen is sufficiently included in a cell, and especially the fat may be used as an energy source only in case of oxidizing within a cell. As described above, if a human being does not provide his cells with oxygen, he does not survive. In process of an internal oxygen metabolism, however, a free radical, called noxious oxygen or active oxygen, is occurred.

The free radical is matter having unpaired electron, which has high reactivity and low lifetime. The normal oxygen remains in a man's body approximately during over 100 second, but the noxious oxygen such as super oxide (O2) or hydroxyl oxygen appears and disappears during from one-millionth second to one-billionth second. This period is very short, but the noxious oxygen causes the peroxide phenomenon of the lipid that is the chief ingredient of the cell membrane, thereby destroying the cell membrane and wrecking the signal transduction system, or destroying a red cell.

As described above, by applying the laser beams of 608 nm to the HPD accumulated in the tumor part, the HPD reacts chemically to generate the active oxygen. The generated active oxygen necrotizes the tumor texture to remove the tumor.

Since the part for applying the laser beams is previously set and the intensity of the laser beams is not harmful to the human body, the normal texture is not received lacerations by the irradiating of the beams. Further, since the active oxygen is occurred only in the part for accumulating the HPD, only the tumor texture on which the active oxygen is occurred by the HPD is necrotized but the normal texture is not affected. Further, since the laser beams don't affect the normal texture, the laser beams can be applied to a wide area beyond the part suspicious of having the tumor. Therefore, the laser beams are applied to even a minute transition tumor texture away from the tumor texture, so that the HPD is accumulated and the active oxygen is occurred in the minute transition texture, thereby necrotizing the minute transition tumor texture not capable of being detected without a close examination.

For curing the tumor, an optical fiber may be used for guiding the laser beams to a general endoscope having an illuminator and a camera. Therefore, since a standard endoscope may be selected by the part to be cured, there is no necessary for making a special endoscope for curing.

As described above, according to the present invention, if the HPD, a kind of blood component, is injected to a patient through his vein, then the HPD is accumulated in the tumor texture but disappeared in the normal texture. After a predetermined time, laser beams of wavelength 608 nm is generated by the laser of YAP:Nd⁺³ and applied to the tumor texture by using an optical fiber or an endoscope, so that the accumulated HPD is reacted chemically to generate the active oxygen for necrotizing the lipid of cell tissue, thereby necrotizing the tumor.

Further, the PDT apparatus according to the present invention generates and irradiates laser beams, which are not harmful to the human body, so that the laser beams can be applied to a wide area including tumor texture and more over a minute transition tumor occurred far away the center of the tumor texture. Therefore, the minute transition tumor texture not capable of being detected without a close examination may be cured and it prevents the normal texture from lacerating due to the irradiation of the laser beams.

What is claimed is:

1. A photodynamic therapy apparatus using a solid-state laser, which comprising:
a laser beam generator 10 for generating laser beams of a predetermined wavelength necessary for curing a tumor, which includes a pulse pumping lamp 12 for generating the light of a predetermined vibration energy and a laser stick 11 for generating laser beams of a predetermined wavelength by using the light from the pulse pumping lamp;
a power supply 18 for providing the laser beam generator with power;
a cooler 17 for preventing the pulse-pumping lamp from overheating;
a first optical resonance-reflecting mirror 7 of convex shape for reflecting and amplifying the laser beams of the predetermined wavelength from the laser beam generator;
a first rotation-reflecting mirror 15 for receiving and reflecting again the laser beams reflected from the first optical resonance;
a barium nitrate crystal (Ba(NO3)2) 13 for receiving the laser beams reflected from the first rotation reflecting mirror and converting the wavelength of the received laser beams by using Ramman Scattering;
a Q-switching lithium fluoride (Lif) crystal 9 for converting again the wavelength of the converted laser beams from the barium nitrate crystal through Q-switching;
a second optical resonance-reflecting mirror 8 for receiving the laser beams from the lithium fluoride crystal and reflecting the laser beams of the non-converted wavelength while outputting the laser beams of the converted wavelength;
a non-linear KTP (Potassium Tytanil Phosphate; KTiOPO4) crystal 6 for outputting the laser beams of the converted wavelength passed from the second optical resonance reflecting mirror at a predetermined wavelength:
a second and a third rotation reflecting mirror 3, 4 for reflecting only the laser beams of a predetermined wavelength in the laser beams from the non-linear KTP crystal;
an absorption energy meter 5 for absorbing a portion of the laser beams passed through the third rotation reflecting mirror 3 and measuring the energy of the absorbed laser beams;
a central processor 16 for receiving the output from the absorption energy meter and controlling the whole operation of the photodynamic therapy apparatus;
a safety shutter 14 for excluding the laser beams reflected from the fifth rotation reflecting mirror under the controlling of the central processor in case that the laser beams measured by the absorption energy meter deviates from a predetermined limit; and
a focusing lens 2 for condensing the light reflected from the third rotation reflecting mirror and irradiating the light to tumor cells through an optical fiber cable directly or with an endoscope.

2. The photo-dynamic therapy apparatus according to claim 1,
wherein, said laser stick is YAP:Nd⁺³ solid-state laser for generating the laser beams of wavelength 1079 nm,
the wavelength of the laser beams generated by said laser beam generator is 1076 nm,
the wavelength of the laser beams converted by said barium nitrate crystal is 1216 nm,
the laser beams converted by said Q-switching lithium fluoride crystal 9 are pulse waves, and
the wavelength of the laser beams output by the non-linear KTP crystal 6 is 608 nm.

3. The photo-dynamic therapy apparatus according to claim 1,
wherein, said pulse pumping lamp is a pulse flash lamp,
said laser stick is an active device, and
said laser beam generator is a pump.

4. The photo-dynamic therapy apparatus according to claim 1,
wherein, said absorption energy meter 5 absorbs one percent of the laser beams passed through the third rotation reflecting mirror 3.

5. A photo-dynamic therapy method using a solid state laser, which comprising the steps of:
injecting HPD (HematoProfilin Derivative), which is a kind of blood component into the tumor texture of a patient;
removing the HPD of the normal texture, while accumulating the HPD of the tumor texture;
applying laser beams of wavelength 608 nm to the tumor texture by using the photodynamic therapy apparatus of the claim 1;
generating active oxygen by reacting the HPD with the laser beams chemically; and
necrotizing the tumor texture by destroying the lipid of the tumor texture with the active oxygen.
